(19) United States
(12) Patent Application Publication
(10) Pub. No.: US 2004/0015238 A1
(43) Pub. Date: Jan. 22, 2004

(54) Shrouds for implants

(76) Inventors: Knute C. Buehler, Bend, OR (US); David C. Kelman, Collierville, TN (US); Jerry L. Jones, Memphis, TN (US); Jeffrey J. Shea, Memphis, TN (US); William B. Austin, Germantown, TN (US); Richard D. Lambert, Cordova, TN (US); Alisha W. Bergin, Southaven, MS (US); Terry W. McLean, Memphis, TN (US)

Correspondence Address: CHIEF PATENT COUNSEL, SMITH & NEPHEW, INC., 1450 BROOKS ROAD, MEMPHIS, TN, 38116, US

(21) Appl. No.: 10/346,316

(22) Filed: Jan. 16, 2003

Related U.S. Application Data
(63) Continuation-in-part of application No. 09/551,375, filed on Apr. 18, 2000, now abandoned.
(60) Provisional application No. 60/186,473, filed on Mar. 2, 2000.

(51) Int. Cl. A61F 2/46

(52) U.S. Cl. 623/22.12; 606/95; 606/102

(57) ABSTRACT

Shrouds for use with an implant in order to allow a surgeon to determine when the implant has been inserted into the femoral canal (or other bone as applicable) a proper distance during implantation. Such a shroud is useful to ensure that the patient's limb after prosthesis implantation is the correct length. The shrouds described have a distal surface which, when properly positioned relative to the implant, indicate proper insertion distance, such as by abutting a portion of the femur upon reaching correct insertion distance. The shrouds may contain a cavity which receives all or part of the neck or taper of the implant in order to position the distal surface correctly. The shrouds described can also help determine correct version of the implant, retain cement in the bone canal or cavity during implantation, pressurize the cement, and protect surfaces of the implant during the implantation.

This application is a continuation-in-part of U.S. patent application Ser. No. 09/551,375, filed on Apr. 18, 2000, having the title “Shrouds for Implants,” which claims priority to U.S. Provisional Application No. 60/186473 filed on Mar. 2, 2000, entitled “Shrouds for Implants,” both of which are incorporated herein by this reference.

### BACKGROUND OF THE INVENTION

The present invention relates to devices for locating implants properly in a patient's bone during implant surgery. Installation of prostheses within the patient's bone structure requires considerable effort and attention. For instance, installation of a femoral implant requires proper preparation not only of the proximal surfaces of the femur, but also proper preparation of femoral canal. Trial reduction usually follows in order to assess bone preparation and to select a properly sized and configured implant. Even with correct preparation of the bone and selection of the proper implant, the implant must be installed in proper position and orientation. In the femoral case, the implant stem must be inserted an appropriate distance into the femoral canal in order to allow the patient's leg to remain the same length as before the surgery. The femoral implant must also be located and oriented correctly in version, that is, in a rotational sense relative to the longitudinal axis of the femur. Location, positioning and orientation of the femoral implant in the femur is even more difficult with cemented femoral implants. There, cement is introduced into the femoral canal and the implant is then introduced into the cement, thereby allowing relative movement between the implant and the bone while the cement hardens.

Collarless implants present additional surgical challenge since they lack the landmarks which otherwise indicates to the surgeon when the implant has been inserted into the femoral canal to the proper extent, with correct positioning and orientation. Implantation of such devices can create tension and complexity during surgery, and requires considerable training, since the surgeon must gauge correct depth, positioning and orientation by eyeball without the usual reference points. Because of the gravity of the downside risk that the patient's leg will be too long or short after such surgery, implantation of collarless cemented implants has historically been reserved to a smaller group of experienced surgeons.

Previous efforts to locate, position and orient implants within the bone during implant surgery, and more particularly, femoral implants, include use of a template which may be placed on or attached to portions of the proximal surface of the femur and through which the implant stem may be inserted. The template thus positions portions of the implant it touches relative to the proximal surfaces of the femur—in both the anterior-posterior direction and the medial-lateral direction. However, such templates do not indicate when the implant has been inserted into the femur an appropriate distance.

Accordingly, there is a need for locating structures, such as shrouds, that can assist surgeons in properly locating implants. It is preferable that such locating structures form a snug fit with the implant so that the implant does not twist or slip within the locating structure. There is a further need for locating structures that can be removed from the implant by a release structure without requiring the locating structure to be manually pulled off of the proximal end of the implant once it has been positioned.

### SUMMARY OF THE INVENTION

The present invention provides locating structures, known as “shrouds,” which fit portions of an implant (or instrumentation attached to the implant) and indicate when the implant has been inserted a proper distance into a patient's bone. One version of such a shroud fits the neck and optionally, the shoulder, of a femoral implant. The implant may, but need not be, collarless and/or adapted for installation with cement. Shrouds according to various embodiments of this invention can feature a cap, a cavity, or any other structure that corresponds at least in part to the shape of the implant neck, including the portions of the taper, in order to hold the neck, whether loosely or tightly, or retain the shroud on the implant. (For purposes of this document, the term “hold” means to assist, however slightly and in whatever manner, releasable or not, whether by deformation of one or both structures or not, in causing two structures, such as the cap or cavity and the implant, to be in position relative to each other. Thus, the shroud can be held to the component, whether implant, instrumentation or other component, adjustably or not, using deformation properties, friction, screws, clamping, welding, integral forming, surrounding the entire neck of the implant, or any other technique for causing two bodies to be located adjacent to each other in whatever manner desired.)

In one embodiment, a frame can connect a cap to a distal surface, defining a flange, which is positioned and oriented to abut portions of the femur when the implant has been inserted a proper distance into the bone. In another embodiment, the shroud itself is an elongated cap-type structure defining a cavity with a base that forms a distal surface. The cavity substantially fully covers the proximal portion of an implant, e.g., a taper and a neck. In other words, this embodiment provides an elongated cavity that snugly receives an implant. The base of the cavity is a distal surface that provides substantially 360° coverage of at least a portion of the neck and that is adapted to abut portions of the femur when the implant has been inserted a proper distance into the bone. This embodiment is particularly useful to prevent cement from seeping up from the cavity toward the inner portion of the shroud and around the neck portion. Instead, it directs cement away from the neck portion.

In addition to the 360° coverage provided by the shroud, shrouds according to various embodiments of this invention may also be provided with a wiper feature at or near the opening of the cavity. This further prevents cement from seeping inside the shroud, between the shroud and the implant.

Shrouds of this invention, by referencing the distal surface to the implant (or its instrumentation), help a surgeon determine proper insertion distance of the implant consistent with pre-operative planning. This feature is particularly useful for collarless hip designs, but use of the invention is not limited to such designs.

As used in this document, “distal surface” means any distal portion of the shroud, such as a flange, a web, a base of a cavity or any other structure, that is adapted to abut a portion of the femur when the implant has been properly positioned. The distal surface may also help to keep cement within the femoral canal by physically impeding flow of the cement outside the canal as the implant is being inserted. The distal surface can also help pressurize the cement which improves surgical outcome by forcing the cement to integrate into the trabecular bone thereby improving the bond between the cement and bone interface.

Certain other embodiments of this invention also provide a release structure, such as a cutting guide groove (or grooves), that allows a practitioner to remove the shroud from the implant once the implant has been properly positioned, without the need to slide the locating structure off of the proximal portion of the implant. The release structure may further be defined by a ridge cap such that the surgeon may run a scalpel or specially designed instrument through the groove, which is an area having a smaller thickness than the remaining portion of the locating structure, to break the ridge cap in order to split the locating structure and “peel” it away from the implant once the implant has been properly positioned. Alternatively, the release structure may be provided such that a practitioner may break or split the shroud apart by pulling it apart and removing it from the implant.

The shroud can also help in evaluating the amount of version in which the stem is placed, which can affect range of motion of the prosthesis. The surgeon can use the shroud, with or without indicia, to compare position of the prosthesis to bony landmarks such as the lesser trochanter. If provided, indicia assist the surgeon to properly align the shroud.

Shrouds according to the present invention can also help protect the neck and shoulder of the implant, including the taper. It is important not to scratch or otherwise deface the taper, which could otherwise affect the fit of the modular femoral head and the corrosion properties of the head and taper interface. Protection of the neck and shoulder also helps improve wear properties of the implant in the event they impinge on the acetabular cup which receives the femoral head.

Another feature that may be particularly useful is to provide the shroud in a material that is at least partially clear or translucent. This allows the surgeon to easily visualize the implant in place within the shroud in order to ensure that implant is properly placed entirely within the shroud.

Accordingly, the present invention includes a shroud for use with an implant including a stem and a neck, the shroud comprising:

(a) an elongated, walled cavity having a shape that corresponds to the neck, the cavity adapted to fit snugly over the neck in order to locate the shroud on the implant;

(b) a distal surface defining a base of the cavity, the distal surface adapted to substantially surround at least a portion of the implant and at least partially abut the patient's bone into which the stem is inserted and assist in determining whether the implant has been inserted into the cavity of the patient's bone a proper distance.

The invention further includes a shroud for use with a femoral implant, the femoral implant including a stem for insertion into a canal of the patient's femur, a neck that includes a taper, and optionally, a shoulder connecting the stem and the neck, the shroud comprising an elongated cap-type structure formed from deformable material and having an internal cavity, preferably an elongated, walled cavity, with a shape that corresponds to the neck, the internal cavity defined at its base by a distal surface adapted to abut a patient's bone in use to assist in determining whether the implant has been positioned and oriented properly, the internal cavity further optionally defining a release structure for removal of shroud after the femoral implant has been appropriately placed.

The shroud can be provided in any desired structure for causing it to be positionable in an adjustable fashion on the implant or on instrumentation for the implant, in order to allow the surgeon to select from a range of options as to proper insertion depth of the implant. Such adjustability can also be helpful in accommodating various sizes of implants.

The shroud can be provided in disposable or non-disposable form. It can be attached to the implant in any desired way with any desired structure, the primary objective of the invention being to provide a structure such as a distal surface that is disposed adjacent to the implant with reference to at least one point on the implant (whether by attachment to the implant or to implant instrumentation) in order to assist in correctly inserting and positioning the implant during surgery.

It is therefore an object of the present invention to provide shrouds for attachment to prostheses in order to indicate proper placement of the prostheses in bone.

It is an additional object of the present invention to provide shrouds for attachment to implants in order to indicate when the implants have been inserted into the bone a proper distance.

It is an additional object of the present invention to provide shrouds for attachment to femoral implants in order to accomplish any or all of the following: to indicate when the implants have been inserted into the bone a proper distance, to indicate proper version, to retain and pressurize cement in the femoral canal, and to protect portions of the implant during surgery.

Other objects, features and advantages of the present invention will become apparent with respect to the remainder of this document.

### BRIEF DESCRIPTION OF THE DRAWINGS

FIG. 1 is an exploded perspective view of a first embodiment of a shroud according to the present invention for a femoral implant, combined with a femoral implant and an acetabular implant.

FIG. 2 is a perspective view of the shroud of FIG. 1 adjacent to the femoral implant.

FIG. 3 is a perspective view of the shroud of FIG. 1 positioned in place on the femoral implant.

FIG. 4 is a perspective view of the shroud of FIG. 1 on the implant which is inserted into the femoral canal.

FIG. 5 is a side elevational view of another shroud according to the present invention on an implant.

FIG. 6 is a top plan view of the shroud of FIG. 5 on the implant.

FIG. 7 is a cross-sectional view of the shroud of FIG. 5, taken on line 7-7 of FIG. 6.

FIG. 8 is an exploded perspective view of a second embodiment of a shroud according to the present invention for a femoral implant, combined with a femoral implant.

FIG. 9 is a perspective view of the shroud of FIG. 8 positioned in place on the femoral implant.

FIG. 10 is a side cross sectional view of the shroud of FIG. 8.

FIG. 11 is a perspective view of an alternate embodiment of a shroud of the present invention.

### DETAILED DESCRIPTION OF THE DRAWINGS

Shrouds of this invention are intended for use with any prosthesis which is to be implanted into a patient's bone, particularly prostheses having a stem and a neck. The shrouds are described below with reference to a femoral implant, but it should be understood that they are usable with any type of implant prosthesis, such as a shoulder implant, knee implant, and so forth. Additionally, although the various features of shrouds 10 and 100 are described separately below, it should be understood that certain features described for shroud 10 may be present on shroud 100, and vice versa.

FIG. 1 shows a first embodiment of a shroud 10 according one embodiment of the present invention exploded from an implant 12. Implant 12 may be a femoral implant which is affixed in the femur with or without bone cement. It may include or exclude a collar at the shoulder, and it may include or exclude porous coated surfaces for bone fixation. In general, implant 12 includes a stem 14, which is generally elongated and adapted structurally for insertion into the femoral canal, a neck 16 which includes structure 18 for receiving a femoral head 20, and a shoulder 22 that connects the stem 14 and the neck 16. The femoral head 20 may be essentially spheroid in shape and contain structure for receipt of companion structure 18 on neck 16. Usually, neck 16 structure 18 is in the form of a taper 24 which in turn includes a proximal surface 26, a relief and a frustoconical surface 30 which may act in Morse taper fashion with respect to cavity 32 in femoral head 20. Cavity 32 is preferably an elongated, walled cavity, such that there are substantially no openings in the walls defining cavity.

The shoulder 22 of implant 12 may but need not contain a collar (unshown) for partial abutment against portions of the femur, but the present invention is particularly well suited for implants 12 without such collars or with collars which may not provide sufficient indication of correct positioning of implant 12 in the femur or retention of cement in the femoral canal.

Femoral implant 12 may therefore be collarless or with a collar, and it may be adapted for cement fixation in the femoral canal or fixation without cement. It may but need not include porous coated surfaces for bone ingrowth. Implant 12 is typically formed of metallic material having the right corrosion resistance, strength and cost parameters. In short, implant 12 can be any desired femoral implant formed according to any desired design with any materials, for fixation in the femoral canal by whatever process or means.

Femoral head 20 may be formed of metallic material, polymeric, ceramic or other desired material. Head 20 fits in an acetabular cup which has been inserted in the acetabulum of the patient that corresponds to the femur having the femoral implant 12. The cup may include a liner such as a polymeric liner 36 to receive head 20. The liner 36 may move in gross motion generally corresponding to a portion of the motion between the torso and the femur (so-called articulation) or the liner 36 may be locked within the cup 34. Cup 34 need not contain a liner such as in instances where the head 20 is formed of polymeric material or in metal-to-metal designs. Cup 34 may be fixed in the patient's acetabulum with or without cement.

As mentioned above, shrouds according to the present invention may also be used for other prostheses, including shoulder implants and acetabular cups 34 for indicating that the prosthesis is inserted a correct depth into the bone, for retaining and/or pressurizing cement during implantation of the prosthesis, for referencing version of the prosthesis, and/or for protecting portions of the implant during surgery. In the acetabular shroud, a flange may be placed circumferentially about the cup in order to indicate correct depth of insertion and orientation of the cup in the bone, to retain and pressurize cement, and/or to protect the cup or portions of it. In the shoulder implant, the shroud could be structured and act similarly to the embodiments disclosed herein.

Surgeons often face difficulty in determining when stem 14 has been inserted a proper distance into the cement-filled femoral canal during implantation. Furthermore, the surgeon can face difficulty in determining whether implant 12 is oriented in version with respect to the femur. The shrouds of this invention are adapted to help the surgeon in either of these determinations, or both of them, as well as to help retain and pressurize cement in the canal during implantation, and for other desired purposes. The structure of the shroud does not matter, generally, so long as it allows shroud to dispose a distal surface, such as a flange or a base of a cavity, near or adjacent to shoulder 22 of implant 12 in whatever manner to reference distal surface in distance and/or angular orientation relative to another part of the structure of the implant 12 (whether by attachment to the implant 12 or instrumentation attached to implant 12) in order to allow distal surface to indicate to the surgeon when the stem 14 has been inserted a proper distance into the femoral canal, or when implant 12 is located correctly in the femoral canal in version.

Distal surface can assume any desired shape. It can be generally collar shaped as are collars on some implants. In the embodiment shown in FIG. 1, distal surface 53 is flange-shaped 50 for abutment against a portion of the femur in order to indicate to the surgeon proper stem insertion distance. The distal surface 53 need not be flat or of any other shape; instead, it can be of any desired shape to abut parts of the femur in order to allow the shroud 10 to indicate when the implant 12 has been inserted to the correct depth in the femoral canal. Flange 50 or distal surface 53 can be larger than the typical collar found on some implants, particularly if shroud 10 functions to retain bone cement in the canal during stem insertion.

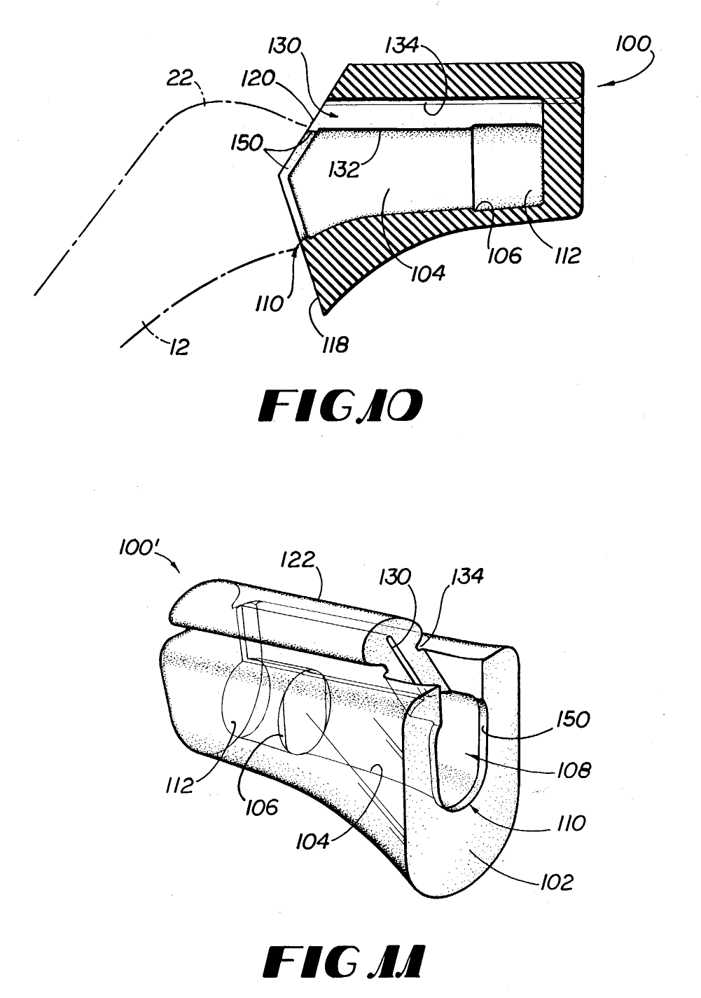

In the embodiment shown in FIG. 8, the distal surface 102 of shroud 100 is provided by a base 110 that is preferably an oblong-shaped distal surface 102. Distal surface 102 provides substantially full (approximately 360°) coverage of at least a portion of the neck 16 and is adapted to abut portions of the femur when the implant 12 has been inserted a proper distance into the bone. For example, as shown in FIG. 9, in use, distal surface 102 surrounds and/or encloses neck 16. Among other things, distal surface 102 prevents cement from seeping up through shroud 100, which is a common occurrence with shrouds that only partially surround the neck. For example, when U-shaped shrouds or shrouds having a U-shaped distal surface are used, cement has been found to seep up and out of the closed end of the “U.” On the other hand, with the oblong-shaped distal surface 102 of shroud 100 that surrounds and/or encloses neck 16, cement is directed to seep outside the oblong “O” because the practitioner can apply pressure on all sides of surface.

Distal surface 102 is formed by base 110 of cavity 104 which allows shroud 100 to at least partially, and preferably completely, surround neck 16 of implant 12 and hold, whether loosely or tightly, the shroud 100 near shoulder 22. Distal surface 102 may, but need not be, oriented in angle in the same way as a collar of a collared implant. It may, for instance, but need not be, oriented generally perpendicular to the longitudinal axis 52 of neck 16 and taper 24. Distal surface 102 may optionally define presentation surface 120 which provides a release mechanism, described below.

An oblong-shaped distal surface 102 and an oblong cavity 104 that correspond to the shape of neck 16 allow the shroud 100 to provide an optimal fit around implant 12. Surface 102 and cavity 104 are adapted to substantially completely surround neck 16 in order to secure the shroud 100 to implant 12 and prevent implant 12 from slipping, twisting or otherwise moving within cavity 104 of shroud 100. Distal surface 102 preferably provides a close fit to neck 16 such that any cement that escapes from the implantation of implant 12 is directed outward, away from cavity 104 and not into shroud 100.

Cavity 104 extends proximally from distal surface 102 and is preferably shaped complementary to the shape of neck 16 and taper 24, although this is not required. Cavity 104 can perform its holding function as long as it defines an opening 108 that fits snugly over and receives neck 16 and taper 24. In a preferred embodiment, cavity 104 defines lip structure 106 (also referred to as a “detent”) corresponding to lip of 28 of the taper 24. Because shroud 100 is preferably formed of a resilient material, lip structure 106 can be formed in cavity 104 such that shroud 100 snugly receives lip 28 of taper 24 and prevents implant 12 from sliding out of shroud 100. This configuration further protects and encloses the taper 24 and neck 16 of implant 12. Additionally, because of the tight fit that is preferably provided, the implant 12 is prevented from slipping, twisting, or otherwise moving within shroud 100.

Alternatively or additionally, cavity 104 may define a tapered section without a lip structure for receiving taper 24 of implant 12. Further, alternatively or additionally, cavity 104 may define a plurality of detents to secure implant in shroud 100. (An embodiment having a plurality of detents is not shown in FIGS. 8-12, but is shown in FIGS. 1-4 in connection with shroud 10. These features are equally applicable for use in connection with shroud 100.) In short, cavity 104 may be of any desired shape and structure to hold or retain any portion of implant 12 (or instrumentation attached to it), preferably a portion of neck 16 of implant 12, in order to reference in position and orientation distal surface 102 with respect to a patient's bone during implantation. Cavity 104 also surrounds and “covers” a portion of taper 24 and neck 16 and/or other portions of the implant 12 to provide additional protection of those members during implantation.

Cavity 104 terminates at base 110. Base 110 is defined by distal surface 102, which may have first and second surfaces that are optionally angled from one another. A first surface 118, an abutment surface, is provided that is adapted to abut the patient's femur in use. Abutment surface 118 may be curved, flat, or otherwise shaped to approximate the natural shape and contour of the a patient's femur or other feature. A second surface, the presentation face 120, at least partially defines a release structure. A release structure is particularly advantageous because once the implant has been positioned, the practitioner may need to remove shroud, but not want to disturb the implant position if, for example, the cement has not set completely and the implant in not yet stable. Additionally, the surgeon may not want to disturb tissue surrounding the implant by applying pressure to pull the shroud off of the neck of the implant.

In one embodiment, release structure is provided by ridge cap 122 and groove 130. Ridge cap 122 defines a section of shroud 100 that allows shroud 100 to be cut away from implant 12 after use. Specifically, ridge cap 122 at least partially defines cutting groove 130 for ease of removal of shroud 100. Cutting groove 130 is further defined by cavity 104 on its opposite surface. Although a single release structure, in this case, cutting grove 130, is described, it should be understood that multiple release structures may be provided as necessary. In addition to allowing shroud 100 to be easily removed from implant 12, release structure also helps prevent the implant for being cut or scratched as shroud is being removed.

Cutting groove 130 (or grooves) allows a practitioner to remove the shroud 100 from the implant once the implant has been properly positioned by using a scalpel or other specially-designed cutting instrument to engage the groove 130. (In some embodiments, specially curved and angled instruments specifically designed for this purpose may be provided. In other embodiments, the surface defining the groove itself, presentation face 120, is angled, so that standard cutting blades may be used with ease.) The material forming the groove 130 is preferably thinner at the groove site, so that once the front edge 132 of the groove in engaged, the application of slight to moderate pressure can split the shroud 100. The surgeon may slide scalpel along font edge 132 of groove 130 and cut groove side 134.

Alternatively, release structure may be a thin portion of open material (not shown), such that a surgeon does not need to use a removal instrument, but can merely “peel” the shroud away after use. Further, release structure may be perforations, scored material, or any other embodiment that allows removal of shroud from implant without actually pulling the shroud off neck of the implant. Release structure may be located anywhere on shroud 100, so long as it allows the practitioner to remove shroud 100 from implant 12. It has been found particularly advantageous to provide at least one release structure, such as groove 130, on the anterior portion of shroud 100. This allows removal with the least amount of maneuvering or pressure to the implant 12.

Shrouds according to various embodiments of this invention may also be provided with a wiper feature 150 at or near the opening 108 of the cavity 104. Wiper 150 may be a small lip or indentation at least partially defining opening 108 that prevents cement from seeping inside shroud, between the shroud and the implant. Wiper 150 is shown surrounding the opening 10, although it should be understood that wiper 150 may traverse only a portion of opening 108. Its main function is provide a barrier to prevent seepage of cement to undesired areas of the shroud.

One way of properly positioning and orienting distal surface relative to implant 12 is shown in FIG. 1. There, cap 54 is structurally configured in size and shape to fit portions of neck 16 of the implant 12. Cap 54 in the embodiment shown in FIG. 1 is configured to be generally cylindrically or frustoconically shaped to receive taper 24 to form a sort of cage about taper 24. Cap 54 need not fit the taper 24 or neck 16 perfectly or even closely; it can correspond to the taper 24 or neck 16 in shape, which means for purposes of this document that cap 54 can have any shape desired to be positionable on or near taper 24 or neck 16 in order to hold the neck 16, taper 24, implant 12, or any portion or portions of those structures (or any part of the instrumentation used with implant 12), in order to reference the distal surface 50 relative to the implant 12.

One advantage which can be provided by the invention is a distal surface or other structure indicating positioning that can be adjustably positioned relative to the implant, in order to give the surgeon a range of options as to how deeply or otherwise how to install the implant in correct position. One way to do this can be using one, two, or preferably more detents 60 which hold relief or lip 28 of the taper 24. More than one detent allows the shroud to be retained on the implant 12 in an adjustable fashion, and thereby to have a degree of control of where the distal surface is positioned relative to the implant 12. Such optional control provided by detents 60 allows the surgeon to select the extent to which the implant 12 will be inserted or otherwise positioned in the femoral canal as indicated by the distal surface. For purposes of this document, the term “detent” means any structure which can hold the shroud relative to the implant or its instrumentation. Detent or detents 60 can be formed, among other ways, of irregularities such as indentations into the surface of cap 54 or cavity 104, protuberances from the surface, or other irregularities, and they can be formed at points on the surface or extend in arcuate fashion around the surface to whatever extent desired.

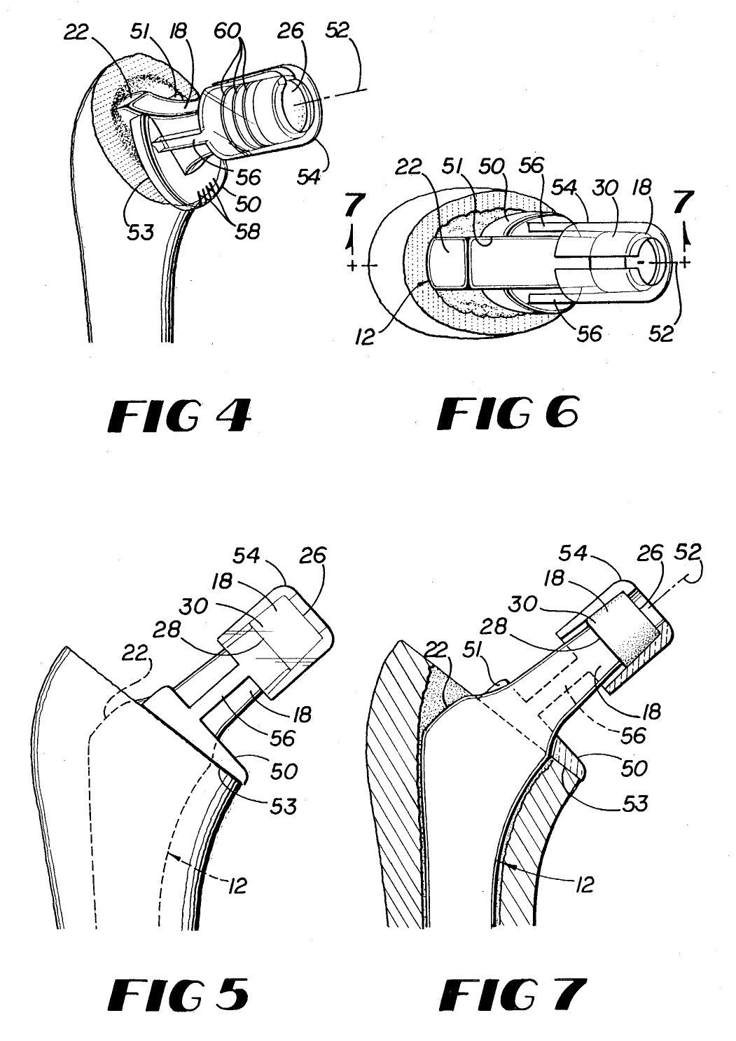

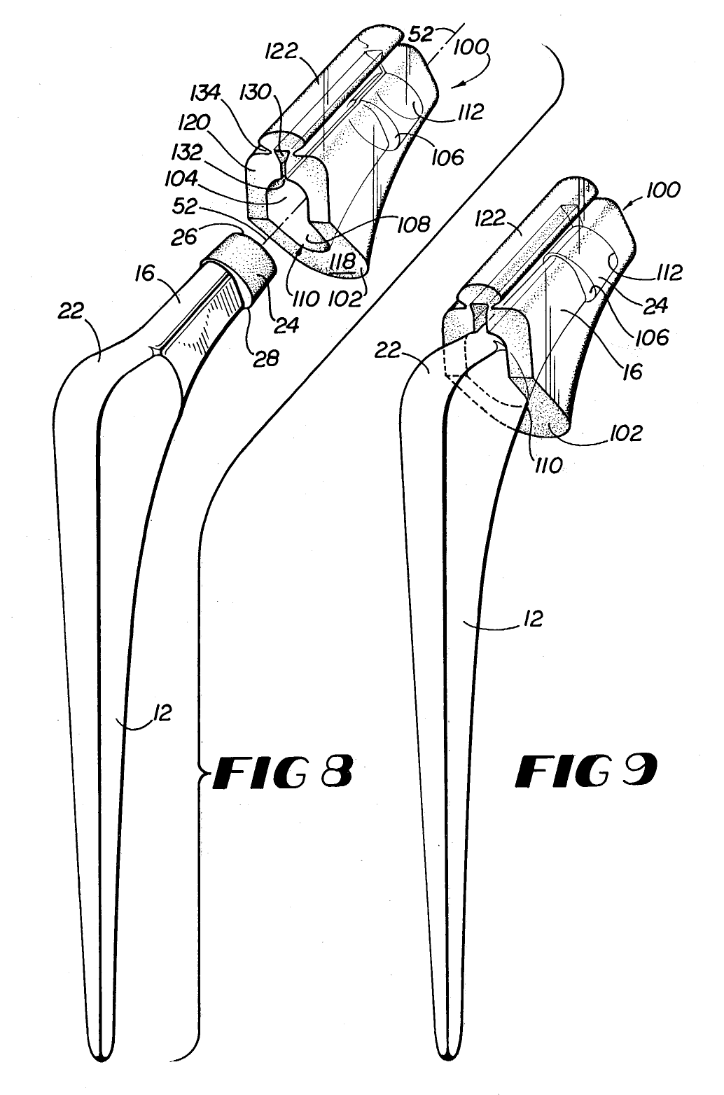

Cap 54 can perform its holding function with or without detents. As shown in FIGS. 4-6, cap 54 need not contain detents 60; instead it can conform to some degree to the shape of the taper 24, with or without partial deformation of the cap 54; in such designs, the cap 54 references the flange 50 (which defines distal surface 53) relative to the implant 12 relative to the proximal surface 26 of the taper 24. The cap 54 could just as easily hold or retain only portions of the proximal surface 26 of taper 24 and relief 28, without holding or retaining much, if any, of the frustoconical surface 30; alternatively, it could hold or retain portions of the proximal surface 26 and the thinner portions of neck 16 located distally of taper 24. In short, cap 54 may be of any desired shape and structure to hold or retain any portion of implant 12 (or instrumentation attached to it), but preferably a portion of neck 16 of implant 12, in order to reference in position and orientation the flange 50. Cap 54 can also surround and “cover” a greater portion of taper 24 and neck 16 and/or other portions of the implant 12 if protection of those members during implantation or at other times is desired.

The shroud can be held to the implant, or any other component such as instrumentation, using any desired technique or structure. Any portion of the shroud can hold any portion of the implant or other component. Again, for purposes of this document, the term “hold” means to assist, however slightly and in whatever manner, releasable or not, whether by deformation of one or both structures or not, in causing two structures, such as the cap and the implant, to be in position relative to each other. Thus, the shroud can be attached or held to the component, whether implant, instrumentation or other component, adjustably or not, using deformation properties, friction, screws, clamping, welding, integral forming, or any other technique for causing two bodies to be located adjacent to each other in whatever manner desired.

For example, as shown in FIGS. 1-7, a frame 56 which may be configured to have any desired structure of any shape, can connect flange distal surface 53, in this case, flange 50, and cap 54 in order to allow flange 50 to be stably positioned relative to cap 54 in distance and orientation. In the embodiment shown in FIG. 1, frame 56 is a set of longitudinal members that span cap 54 and flange 50. It could just as easily be argued that part of the frame 56 is actually part of the cap 54 and part of it is part of flange 50; the only thing that matters is that cap 54 is connected in some manner, directly or indirectly, to flange 50 distal surface 53, and any structure between portions of the two can be considered, if desired, a frame 56.

If frame 56 is a recognizable structure, it can assume the configuration of a partially cylindrical member, a number of longitudinal members, a cage, or otherwise be shaped and structured as desired to position flange 50 relative to cap 54. However, there need not be a frame 56; cap 54 can simply be connected to distal surface directly, similar to the embodiment shown in FIG. 8-10.

For example, as shown in FIGS. 8-10, distal surface 102 may be directly and integrally formed with cavity 104, without the use of frame 56. This embodiment is preferred because it allows shroud 100 to fit like a snug “glove” over implant 12 to support implant 12 and prevent it from twisting within shroud 100. This embodiment also helps prevent cement from seeping around neck 16 because distal surface 102 assists in pushing cement away from implant 12.

An alternate embodiment of shroud 100′ is shown in FIG. 11. In this embodiment, shroud 100′ includes distal surface 102 and groove 130 at the base portion of shroud 100′. This embodiment may be particularly helpful in removing cement from the base 110 of cavity 104.

Shrouds according to this invention may be formed of any desired material, such as polymeric or silicone material which can deform at least partially to be placed over portions of neck 16 of implant 12 in holding or retaining relationship. For example, shroud 100 (or other shrouds according to various embodiments of this invention) is preferably of a resilient material such that opening 108 of cavity 104 can be placed over neck 16 with upper ceiling 112 of cavity 104 interfacing with, abutting, or otherwise holding shroud 100 relative to the proximal surface 26 of the taper 24, as shown in FIG. 9. Preferably, cavity 104 conforms to some degree to the shape of taper 24. Other materials as desired may be employed to form shroud 100, and deformable to a greater or lesser extent depending upon whether shroud 100 is desired to be used with a number of different sizes of implant 12.

Additionally, shroud 100 is optionally formed of a material that is at least partially clear, translucent, or otherwise see-through. This allows the surgeon to easily visualize the implant 12 in place within the shroud 100 in order to ensure that implant 12 is properly placed entirely within the shroud. Specifically, it is preferable that proximal portion 26 of taper 24 abut upper ceiling 112 of cavity 104 of shroud 100. This means that the implant 12 is fully engaged within the shroud 100 and no substantial movement will be allowed due to the snug fit. A see-through shroud allows the surgeon to visualize and ensure this configuration is proper.

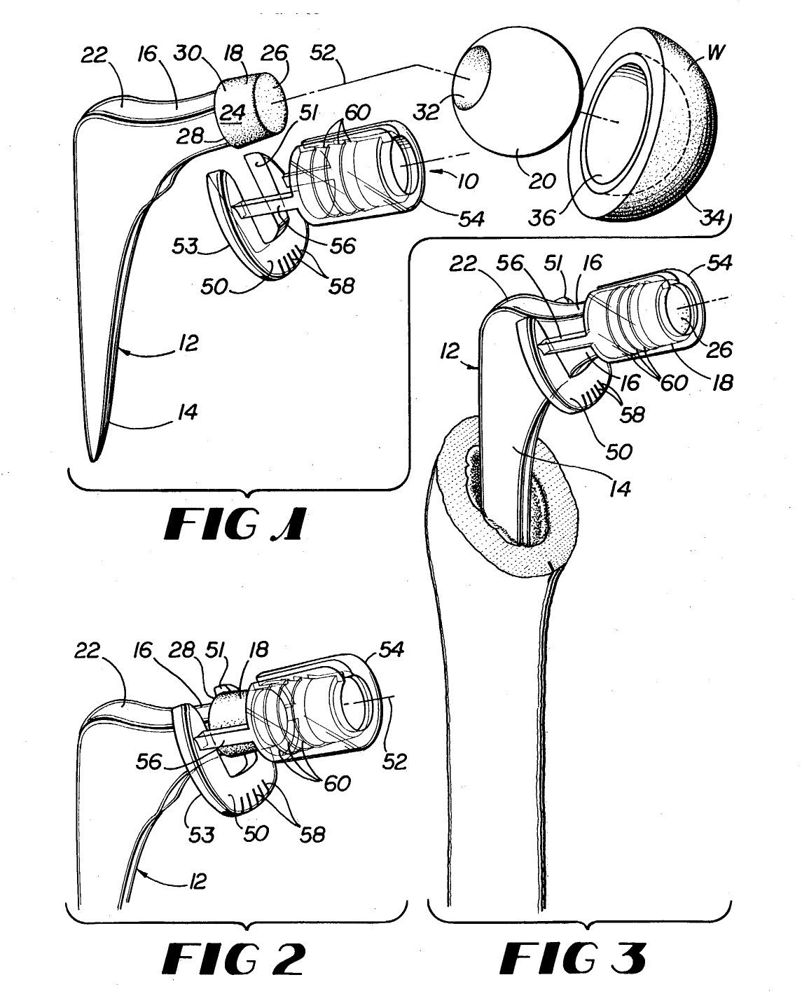

FIG. 2 shows shroud 10 in the process of being placed on implant 12. FIG. 3 shows the shroud 10 in place on the implant, and FIG. 4 shows the shroud 10 inserted in the femoral cavity. The flange 50 distal surface 53 is thus disposed, distanced and oriented relative to the cap 54 and the implant neck 16 so as to abut part of the femur when the implant 12 is properly located in the patient's femur, thereby indicating proper location of the implant 12 in the femur. Such location may include stem insertion distance, version, or and/or desired angles or parameters. It is possible to provide various shrouds 10 having frames 56 of different lengths in order to provide the most options to the surgeon.

FIG. 8 shows shroud 100 in the process of being placed on implant 12, and FIG. 9 shows shroud 100 in place on the implant. The distal surface 102 is thus disposed, distanced and oriented relative to the cavity 104 and the implant neck 16 so as to abut part of the femur when the implant 12 is properly located in the patient's femur, thereby indicating proper location of the implant 12 in the femur. Such location may include stem insertion distance, version, or and/or desired angles or parameters. It is possible to provide various shrouds 100 having cavities 104 of various lengths and widths in order to provide the most options to the surgeon. Such shrouds 100 may be used for with different sized implants or to provide different insertion depths while positioning a single implant.

Indicia 58 may but need not be included on distal surface 50 or 102 or other portions of the shroud 10, 100 to show version or other desired angles or parameters. Such information can be shown simply by orientation of the distal surface 50 or 102, or orientation of other parts of the shroud 10, 100.

In use, the surgeon prepares the proximal portion and canal of a femur of a patient in a conventional manner, using broaches, reamers, instruments to shape the proximal portion of the femur, and other devices and instruments as desired. She then performs trial reduction in order to select the properly configured and sized femoral implant 12, and otherwise to gauge dimensions, angles and other parameters that matter in correct installation and implantation, and otherwise to prepare the femur for the implant. She then introduces cement into the canal of the femur and may then place the selected femoral implant 12 with shroud 10, 100 according to the present invention into the femoral canal with cement (the shroud 10, 100 can be placed after insertion or partial insertion of implant 12 if desired). The correctly positioned distal surface 53, 102 may help the surgeon to do any or all of the following, among other things: (a) determine when the stem 14 of implant 12 is inserted in the femoral canal a proper distance; (b) determine correct version of implant 12; (c) retain cement in the femoral canal; (d) pressurize the cement in the canal; and (e) protect portions of the neck 16 and taper 24.

Using the shroud 10, 100 to help properly locate the femoral implant 12 in the femur, the surgeon allows the cement at least partially to harden in order to affix or retain the femoral implant 12 in the femur. The surgeon then removes the shroud 10, 100 and places the femoral head 20 on the implant.

As to the acetabular components, the surgeon prepares the acetabulum of the patient that corresponds to the femur into which the femoral implant 12 has been fixed. Such preparation again typically involves broaches, instrumentation and other devices in order to prepare the acetabulum to receive a cemented or cementless cup, which can but need not include porous bone ingrowth coating, screws, pegs or other affixation devices. The surgeon installs an acetabular cup 34 with or without liner 36 and then orients the femoral head 20 in the cup 34 or liner 36. After determining that positioning of the prosthesis is satisfactory both statically and dynamically, the surgeon completes the operation.

The chief aim of shrouds according to the present invention can therefore be seen to provide a locating structure such as a distal surface that is positioned in a known way relative to the implant 12 (or instrumentation attached to implant 12), in order to allow the surgeon to determine when the implant 12 has been inserted a proper distance into the femur of the patient during the implantation. So long as that result is obtained, any structure which functions in any way to accomplish such positioning of the distal surface structure is within the scope of the present invention. As was stated above, the shroud can also, but need not, accomplish other results such as indicate correct version of the implant, retain cement in the femoral canal during implantation, pressurize the cement, and/or protect portions of the implant neck or taper.

What is claimed is:

1. A shroud for an implant, wherein the implant includes a stem with a neck, the shroud comprising:
(a) an elongated, walled cavity having a shape that corresponds to the neck, the cavity adapted to fit snugly over the neck in order to locate the shroud on the implant;
(b) a distal surface defining a base of the cavity, the distal surface adapted to substantially surround at least a portion of the implant and at least partially abut the patient's bone into which the stem is inserted and assist in determining whether the implant has been inserted into the cavity of the patient's bone a proper distance.

2. The shroud of claim 1, further comprising a release structure to allow removal of shroud after use.

3. The shroud of claim 2, wherein the release structure comprises a cutting groove adapted to allow a cutting instrument to split the shroud for its removal from the implant after implantation.

4. The shroud of claim 3, wherein the cutting groove comprises a section of material having a thickness less than other portions of the shroud, such that it can be easily cut away.

5. The shroud of claim 2, wherein the release structure is a thin portion of open material such that the shroud can be peeled away from the neck after implantation of the stem.

6. The shroud of claim 1, wherein the cavity and the distal surface are integrally formed.

7. The shroud of claim 1, wherein the distal surface comprises angled first and second surfaces, the first surface adapted to abut the patient's bone and the second surface defining a release structure.

8. The shroud of claim 1, wherein the shroud is formed of a deformable material in order to accommodate different sized implants.

9. The shroud of claim 1, wherein the shroud is formed of polymeric material.

10. The shroud of claim 1, wherein the shroud is formed of an at least partially clear, translucent, or see-through material in order to allow visualization of the proper positioning of the shroud on the implant.

11. The shroud of claim 1, wherein the neck includes a taper with a lip, and the cavity comprises one or more lip structures adapted to hold part of the lip in order to provide the surgeon a range of options in positioning the implant.

12. The shroud of claim 1, wherein the shroud holds the implant removably.

13. The shroud of claim 1, wherein the implant is adapted to be retained in the patient's bone with bone cement, and the distal surface is adapted in shape to help retain the bone cement in the cavity of the patient's bone when the implant is properly inserted into the cavity.

14. The shroud of claim 1, wherein the shroud further comprises indicia in order to assist in properly locating the implant in the cavity of the patient's bone.

15. The shroud of claim 1, wherein the shroud further comprises a wiper to prevent seepage of cement between the shroud and the implant.

16. A shroud for use with a femoral implant, the femoral implant including a stem for insertion into a canal of the patient's femur, a neck that includes a taper, and a shoulder connecting the stem and the neck, the shroud comprising:
an elongated cap-type structure formed from deformable material and having an internal cavity with a shape that corresponds to the neck, the internal cavity defined at its base by a distal surface adapted to abut a patient's bone in use to assist in determining whether the implant has been positioned and oriented properly, the internal cavity further defining an optional release structure for removal of shroud after the femoral implant has been appropriately placed.

17. The shroud of claim 16, wherein the optional release structure is present and allows removal of shroud after use.

18. The shroud of claim 17, wherein the release structure comprises a ridge cap and a cutting groove adapted to allow a cutting instrument to split the shroud for its removal from the implant after implantation.

19. The shroud of claim 16, wherein the shroud is formed of a deformable material in order to accommodate different sized implants.

20. The shroud of claim 16, wherein the shroud is formed of an at least partially clear, translucent, or see-through material in order to allow visualization of the proper positioning of the shroud on the implant.

21. The shroud of claim 16, wherein the shroud contains indicia in order to assist in properly locating the implant in the cavity of the patient's bone.

22. The shroud of claim 16, wherein the shroud further comprises a wiper to prevent seepage of cement between the shroud and the implant.

23. A process for installing a femoral implant in a patient, comprising:
a. preparing the proximal portion and canal of a femur of the patient;
b. performing trial reduction in order to select the femoral implant for the femur,
c. introducing cement into the canal of the femur,
d. introducing the femoral implant into the canal of the femur, the implant including a stem for insertion into the canal of the femur, a neck for receiving a femoral head, and a shoulder connecting the stem and the neck;
e. connecting to the femoral implant a shroud, the shroud comprising:
(i) an elongated, walled cavity having a shape that corresponds to the neck, the cavity adapted to fit snugly over the neck in order to locate the shroud on the implant; and
(ii) distal surface defining a base of the cavity, the distal surface adapted to substantially surround at least a portion of the implant and at least partially abut the patient's bone into which the stem is inserted and assist in determining whether the implant has been inserted into the cavity of the patient's bone a proper distance,
f. properly locating the femoral implant in the femur using the shroud;
g. allowing the cement at least partially to harden in order at least partially to retain the femoral implant in the femur;
h. removing the shroud; and
i. placing a femoral head on the implant.

24. A method of performing a hip implant in a patient, comprising:
a. preparing the proximal portion and canal of a femur of the patient;
b. performing trial reduction in order to select the femoral implant for the femur;
c. introducing cement into the canal of the femur,
d. introducing the femoral implant into the canal of the femur, the implant including a stem for insertion into the canal of the femur, a neck for receiving a femoral head, and a shoulder connecting the stem and the neck;
e. connecting to the femoral implant a shroud, the shroud comprising:
(i) an elongated, walled cavity having a shape that corresponds to the neck, the cavity adapted to fit snugly over the neck in order to locate the shroud on the implant; and
(ii) distal surface defining a base of the cavity, the distal surface adapted to substantially surround at least a portion of the implant and at least partially abut the patient's bone into which the stem is inserted and assist in determining whether the implant has been inserted into the cavity of the patient's bone a proper distance;
f. properly locating the femoral implant in the femur using the shroud;
g. allowing the cement at least partially to harden in order at least partially to retain the femoral implant in the femur;
h. removing the shroud;
i. placing a femoral head on the implant;
j. preparing an acetabulum of the patient that corresponds to the femur with the implant;
k. installing an acetabular cup in the acetabulum,
l. placing the femoral head in proper position with respect to the acetabular cup; and
m. completing surgical operations.

25. A femoral implant product, comprising:
1. a femoral implant which includes a stem for insertion into a canal of the patient's femur, a neck for receiving a femoral head, a shoulder connecting the stem and the neck, and a femoral head; and
2. a shroud comprising:
(i) an elongated, walled cavity having a shape that corresponds to the neck, the cavity adapted to fit snugly over the neck in order to locate the shroud on the implant; and
(ii) distal surface defining a base of the cavity, the distal surface adapted to substantially surround at least a portion of the implant and at least partially abut the patient's bone into which the stem is inserted and assist in determining whether the implant has been inserted into the cavity of the patient's bone a proper distance;

26. A product according to claim 25, in which the implant neck features a taper and the cavity covers at least part of a proximal surface of the taper and at least part of tapered surfaces of the taper in order to locate the shroud on the implant and to protect surfaces of the taper.

27. A product according to claim 25, in which the implant neck includes a taper having a lip and the cavity includes at least one detent or lip structure for holding at least part of the lip.

28. A product according to claim 25, in which the implant neck includes a taper and the cavity includes a plurality of detents for holding at least part of the taper in order to provide the surgeon a range of options in positioning the implant.

29. A product according to claim 25, in which the distal surface is generally oblong in shape and features a generally flat distal surface, the oblong shape generally accommodating portions of the implant shoulder, the generally flat distal surface adapted to abut portions of the femur.

30. A product according to claim 29, in which the generally flat oblong surface of the shroud distal surface is adapted to help retain bone cement in the canal of the patient's femur when the implant is inserted into the femur.

31. A product according to claim 25, in which the distal surface is adapted to hold portions of the implant shoulder.

32. A product according to claim 25, in which the shroud contains indicia in order to indicate proper orientation of the implant in the patient's femur.

33. A product according to claim 32, in which the indicia are located on the distal surface of the shroud in order indicate proper rotational orientation of the implant in the patient's femur.

34. A femoral implant product comprising:
1. a femoral implant which includes a stem for insertion into a canal of the patient's femur, a neck that includes a taper, a shoulder connecting the stem and the neck and a femoral head that connects to the neck; and
2. a shroud comprising:
(i) an elongated, walled cavity having a shape that corresponds to the neck, the cavity adapted to fit snugly over the neck in order to locate the shroud on the implant; and
(ii) distal surface defining a base of the cavity, the distal surface adapted to substantially surround at least a portion of the implant and at least partially abut the patient's bone into which the stem is inserted and assist in determining whether the implant has been inserted into the cavity of the patient's bone a proper distance.
